(19) United States
(12) Patent Application Publication
(10) Pub. No.: US 2004/0185170 A1
(43) Pub. Date: Sep. 23, 2004

(54) Method for coating drug-containing particles and formulations and dosage units formed therefrom

(76) Inventors: Shubha Chungi, Sharon, MA (US); Theodore L. Iorio, Millis, MA (US)

Correspondence Address: REED & EBERLE LLP, 800 MENLO AVENUE, SUITE 210, MENLO PARK, CA, 94025, US

(21) Appl. No.: 10/394,552

(22) Filed: Mar. 21, 2003

(51) Int. Cl. A61K 9/20

(52) U.S. Cl. 427/2.14

(57) ABSTRACT

A method for coating drug-containing particles is provided comprising the steps of volatilizing a coating material and condensing the volatilized coating material onto a plurality of drug-containing particles. The method can be carried out without the use of solvents. Coated drug-containing particles as well as formulations and dosage forms containing coated particles are also described. A method for treating patients using drug-containing particles is provided as well.

### TECHNICAL FIELD

This invention relates generally to methods for coating drug-containing particles, granules, pellets, and related drug-containing units. The coated particles exhibit beneficial and useful properties including, but not limited to, masking the taste of the drug, protecting the drug or other particle components from degradation, and increasing the ability of the particles to flow during processing of the particles into, for example, dosage forms such as tablets. In addition, the invention relates to compositions and dosage forms, e.g., tablets, made from the coated particles, as well as to methods for treating a patient.

### BACKGROUND

The pharmaceutical industry has long practiced the technique of coating drug-containing particles for a variety of purposes. For example, U.S. Pat. No. 5,601,761 to Hoffman et al. describes the improved stability associated with drugs that have been coated. U.S. Pat. No. 4,925,675 to Giannini et al. describes the enhanced ability to flow for seeds having a coating comprised of the drug erythromycin and a binder. The ability to mask the taste of drugs by coating drug-containing granules is described in U.S. Pat. Nos. 4,851,226 to Julian, 5,529,783 to Burke et al., 5,460,825 to Roche et al., and 5,489,436 to Hoy et al. In addition, the practice of coating drug-containing particles has been used to modify the release of the drug in vivo.

Generally, drug-containing particles, e.g., granules obtained from conventional granulation techniques, are coated using a process known as “air suspension coating.” According to this conventional technique, particles are fed into a vertical cylinder and are supported by a column of gas, typically air, entering from the bottom of the cylinder. As the particles move throughout the cylinder, a coating solution is introduced into the cylinder, which is also carried throughout the cylinder by the column of air. In this way, the coating solution comes into contact with each of the particles, thereby coating each particle with the coating solution. After a sufficient time has passed, introduction of the coating solution is stopped, and the particles are allowed to dry. The coating solution is often comprised of beeswax, for example, or a cellulosic material such as ethylcellulose. See Pharmaceutical Dosage Forms, Fifth Edition, Ansel et al., Lea & Febiger, Philadelphia, 1990.

A significant drawback to conventional coating processes is their high dependence on organic solvents. The solvent serves to solubilize the coating material for application to the drug-containing particle, whereupon the solvent is then allowed to evaporate away, leaving only the coating material on the drug-containing particle. First, organic solvents are costly, both in terms of acquisition and disposal. Further, by evaporation into the air, solvents, and in particular toxic solvents, have a deleterious impact upon the environment. Given their volatility, solvents are often extremely explosive and require special safety precautions in order to avoid risk to human life. Additionally, some organic solvents, such as methylene chloride, are known carcinogens, and their use should be avoided whenever possible. Although aqueous-based film-coating solutions have been suggested, these solutions present drawbacks of their own, such as slow evaporation. See Ansel et al., supra.

Thus, there is a need in the art to provide a means to coat drug-containing particles without resorting to the use of organic solvents, which are often costly and/or toxic. The present invention addresses both this and other needs in the art by describing a method for volatilizing a coating material and allowing the volatilized coating material to contact the drug-containing particle.

### SUMMARY OF THE INVENTION

One aspect of the invention relates to a method for coating drug-containing particles, wherein the method comprises the steps of volatilizing a coating material and condensing the volatilized coating material onto a plurality of drug-containing particles.

Another aspect of the invention pertains to such a method wherein the coating material is selected from the group consisting of long-chain fatty acids, long-chain fatty alcohols, long-chain fatty esters, long-chain fatty amines, long-chain fatty amides, bile salts, and surfactants.

Yet another aspect of the invention relates to such a method wherein the coating material is a long-chain fatty acid or a long-chain fatty alcohol.

Another aspect of the invention is to provide such a method wherein the coating material is selected from the group consisting of stearic acid, cetyl alcohol, or stearyl alcohol.

Still another aspect of the invention is to provide such a method that is free from solvents.

Yet another aspect of the invention relates to a composition of matter comprising a drug-containing particle having at least one layer of coating material.

Still another aspect of the invention pertains to a pharmaceutical formulation comprising drug-containing particles having at least one layer of coating material.

Another aspect of the invention relates to a dosage form comprising drug-containing particles having at least one layer of coating material.

Yet another aspect of the invention pertains to a method for treating a patient that comprises administering a dosage form containing a therapeutically effective amount of a pharmaceutical formulation that comprises a plurality of drug-containing particles having at least one layer of coating material.

Additional aspects, embodiments, advantages, and novel features of the invention will be set forth in part in the description that follows, and in part, will become apparent to those skilled in the art upon examination of the following, and may be learned by practice of the invention.

In one embodiment, a method is provided comprising the steps of volatilizing a coating material and condensing the volatilized coating material onto a plurality of drug-containing particles. Volatilization of the coating material is carried out by heating the coating material above its volatilization temperature and/or by decreasing the pressure surrounding the coating material. Upon contact with a drug-containing particle, the volatilized coating material condenses onto the particle, which has a lower temperature than the volatilized coating material. Maintenance of the drug-containing particle below the volatilization temperature of the coating material is performed using any suitable technique, e.g., placing temperature regulating means around the container that houses the drug-containing particles, contacting the drug-containing particles with relatively cool air, cooling or freezing the drug-containing particles prior to contacting them with the volatilized coating material, and so forth.

Any coating material may be used in the present method so long as the coating material volatilizes under the appropriate pressure and temperature conditions. Suitable coating materials include, without limitation, those selected from the group consisting of long-chain fatty acids, long-chain fatty alcohols, long-chain fatty esters, long-chain fatty amines, long-chain fatty amides, bile salts, and surfactants. Preferred coating materials include long-chain fatty acids (e.g., stearic acid), long-chain fatty alcohols (e.g., cetyl alcohol and stearyl alcohol), long-chain fatty esters, long-chain fatty amines, and long-chain fatty amides. The coating on the drug-containing particles is useful for, among other purposes, providing a desired release profile in vivo, masking the taste of unpalatable drugs, stabilizing the drug or other component in the particle to degradation, and enhancing the ability of the particles to flow. Advantageously, the entire coating method is expedient and does not require the use of solvents. Furthermore, the invention is not limited with respect to the particular type of drug-containing particle used, since all types of drug-containing particles (including, for example, granules, beads, and pellets) can be coated using the present method.

In another embodiment, a composition of matter is provided wherein a drug-containing particle has at least one layer of a coating material. In contrast to liquid-based methods of coating, the present method provides the ability to apply a thinner coat, given that the coating material is in a gaseous, and therefore, in an extremely less dense state.

In one embodiment, the particle has a monolayer comprised of a plurality of coating molecules having an average molecular length of no more than 1000 angstroms, wherein the monolayer has a thickness of less than about ten times the average molecular length of the coating molecules.

In another embodiment, the particle has at least one layer to improve flow properties that has a thickness of up to 10,000 angstroms. In another embodiment, the particle has at least one taste masking layer that has a thickness of up to 500,000 angstroms and in yet another embodiment, the particle has at least one functional coating layer that has a thickness of up to 2,000,000 angstroms.

In providing another embodiment of the invention, a pharmaceutical formulation and dosage form are disclosed comprising a plurality of the coated drug-containing particles described herein. The formulation can be directly administered to a patient or may be further processed into a suitable dosage form. Preferred dosage forms include, without limitation, tablets made from, at least in part, the formulation described herein, powders comprising the described formulations, and capsules housing the formulation described herein.

In another embodiment, the invention provides a method for treating a patient in which a dosage form is administered that contains a therapeutically effective amount of a pharmaceutical formulation comprised of a plurality of drug-containing particles described herein.

### BRIEF DESCRIPTION OF THE DRAWINGS

FIG. 1 is a schematic illustration of a coating pan system for coating drug-containing particles.

FIG. 2 is a schematic illustration of a rotating granulator system for coating drug-containing particles.

FIG. 3 is a schematic illustration of a tumble-blender system for coating drug-containing particles.

FIG. 4 is a schematic illustration of a fluid bed coater for coating drug-containing particles.

FIG. 5 is a schematic illustration of a modification of the fluid bed coater of FIG. 4.

### DETAILED DESCRIPTION OF THE INVENTION

Before describing the present invention in detail, it is to be understood that this invention is not limited to particular coating materials or drugs, as such may vary. It is also to be understood that the terminology used herein is for the purpose of describing particular embodiments only, and is not intended to be limiting.

It must be noted that, as used in this specification and the appended claims, the singular forms “a,” “an,” and “the” include plural referents unless the context clearly dictates otherwise. Thus, for example, reference to “a coating material” includes a coating material as well as a combination of two or more coating materials, reference to “a drug” includes a single drug as well as combinations of two or more drugs, and the like.

In describing and claiming the present invention, the following terminology will be used in accordance with the definitions set forth below.

The terms “drug,” “active agent,” and “pharmacologically active agent,” are used interchangeably herein to refer to a chemical compound that induces a desired pharmacological and/or physiological effect. The terms also encompass pharmaceutically acceptable, pharmacologically active derivatives of those active agents specifically mentioned herein, including, but not limited to, salts, esters, amides, prodrugs, active metabolites, analogs, and the like. When the terms “drug,” “active agent,” and “pharmacologically active agent” are used, then, it is to be understood that the applicant intends to include the active agent per se in addition to pharmaceutically acceptable, pharmacologically active salts, esters, amides, prodrugs, metabolites, analogs, etc.

The term “drug-containing particle” refers to any composition of matter that contains or is associated with a drug. The term includes a homogeneous particle consisting only of the drug, as well as heterogeneous and admixed compositions comprising the drug. The drug-containing particle is often, although not necessarily, a granule, bead, or pellet.

By “pharmaceutically acceptable carrier” is meant a material or materials that are suitable for drug administration and not biologically or otherwise undesirable, i.e., that may be administered to an individual along with an active agent without causing any undesirable biological effects or interacting in a deleterious manner with any of the other components of the pharmaceutical formulation in which it is contained.

Similarly, a “pharmacologically acceptable” salt, ester, or other derivative of an active agent as provided herein is a salt, ester, or other derivative that is not biologically or otherwise undesirable.

As provided herein, the terms “effective amount” or “therapeutically effective amount” of an agent are intended to mean a nontoxic, yet sufficient, amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective amount” in any individual case may be determined by one of ordinary skill in the art using routine experimentation.

The terms “treating” and “treatment” are used herein to refer to a reduction in severity and/or frequency of symptoms, elimination of symptoms and/or underlying cause, prevention of the occurrence of symptoms and/or their underlying cause, and improvement or remediation of damage.

The terms “condition,” “disease,” and “disorder” are used interchangeably herein to refer to a physiological state that can be prevented or treated by administration of a pharmaceutical formulation as described herein.

The term “patient,” as in treatment of “a patient,” refers to a mammalian individual afflicted with or prone to a condition, disease, or disorder as specified herein, and includes both humans and animals.

“Optional” or “optionally” means that the subsequently described event or circumstance may or may not occur, and that the description includes instances where the said event or circumstance occurs as well as instances where it does not. For example, reference to an “optional pharmaceutically acceptable carrier” in a formulation means that such a carrier may or may not be present, and the description includes formulations wherein a carrier is present and formulations wherein a carrier is not present.

The coating method comprises volatilizing a coating material and condensing it onto a plurality of drug-containing particles. Advantageously, the present method for coating drug-containing particles is carried out in the absence of solvents such as methylene chloride. In addition, any drug-containing particle can be coated using the present method. In preferred embodiments, the drug-containing particle is in the form of a granule, bead, or pellet. The coating can also serve to mask unpleasant tastes, serve a functional purpose such as providing a means for controlling the rate of release of the active agent, and/or enhance the flow properties of the particle during processing.

The drug-containing particles are solid in nature and, as indicated above, can consist of the drug itself, or the drug combined with one or more additional components. Such particles are available from commercial suppliers like Sigma Corp. (St. Louis, Mo.). In addition, the drug-containing particles can be prepared using conventional pharmaceutical techniques. For example, a drug can be molded with a pharmaceutical carrier or an excipient in order to prepare a drug-containing particle. In addition, granules, a specific type of drug-containing particle, can be prepared by moistening a drug-containing powder and passing the moistened powder through a screen with a mesh size that will produce the desired granules, and thereafter, allowing the granules to dry. Other types of drug-containing particles are beads and pellets, wherein, for example, the drug is coated onto a substrate by contacting the substrate with a solution of the drug and allowing the solvent to evaporate. Drugs can also be located within the bead via coating a drug core with a suitable material. Generally, the difference between beads and pellets lies in their shape; beads are generally spherical, while pellets possess an oblong shape.

The invention also contemplates a drug-containing particle having one ore more layers of a coating material, which may be the same or different. In contrast to liquid-based methods of coating, the present invention provides the ability to apply a thinner coat, given that the coating material is in a gaseous, and therefore, in an extremely less dense state.

In one embodiment, the particle is coated with at least one layer having a thickness of up to 2,000,000 angstroms. Exemplary layers having this thickness include functional coating layers such as release rate controlling layers. In another embodiment, the particle is coated with at least one layer having a thickness of up to 500,000 angstroms. Exemplary layers having this thickness include taste masking layers. In yet another embodiment, the particle can be coated with at least one layer having a thickness of up to 10,000 angstroms. Exemplary layers having this thickness include layers to improve flow properties. The particles may also be coated with at least one layer that is comprised of a plurality of coating molecules having an average molecular length of no more than 1000 angstroms, and preferably no more than 500 angstroms. This layer preferably has a thickness of less than about ten times the average molecular length of the coating molecules, and preferably less than 5, more preferably less than two times the average molecular length of the coating molecules. It is also understood that the particles may also have a combination of any of the aforementioned layer types.

Any coating material can be used to coat the drug-containing particle, provided that the coating material will volatilize under suitable pressure and temperature conditions. Although the invention is not limited with respect to the particular coating material used, preferred coating agents are lipidic materials. Preferred lipidic materials are those selected from the group consisting of long-chain fatty acids, long-chain fatty alcohols, long-chain fatty esters, long-chain fatty amines, long-chain fatty amides, bile salts, surfactants, and combinations thereof. Particularly preferred coating materials include long-chain fatty acids, long-chain fatty alcohols, long-chain fatty esters, long-chain fatty amines, and combinations thereof, with long-chain fatty acids and long-chain fatty alcohols being most preferred. Specific examples of coating materials include, by way of illustration and not limitation, long-chain fatty acids (saturated, unsaturated, and polyunsaturated), fatty alcohols (saturated, unsaturated, and polyunsaturated), fatty esters, long-chain fatty amines and amides, bile salts, anionic surfactants, and cationic and amphoteric surfactants, examples of which are provided below.

Suitable long-chain fatty acids (saturated, unsaturated, and polyunsaturated) include, but are not limited to, arachidic acid (n-eicosanoic acid), arachidonic acid, behenic acid (docosanoic acid), capric acid (n-decanoic acid), caproic acid (n-hexanoic acid), caproleic acid (9-decenoic acid), caprilic acid (n-octanoic acid), docosadienoic acid, docosahexaenoic acid, docosapentaenoic acid, eicosadienoic acid, eicosahexaenoic acid, eicosapentaenoic acid, eicosatrienoic acid, elaidic acid (trans-9-octadecanoic acid), eleosteroic acid, erucic acid (13-docosenoic acid), heneicosanoic acid, heptacosanoic acid, heptadecanoic acid, heptanoic acid, hexacosanoic acid, isostearic acid, lauric acid (n-dodecanoic acid), lignoceric acid (n-tetracosanoic acid), linoleic acid, α-linolenic acid, γ-linolenic acid, myristic acid (n-tetradecanoic acid), myristoleic acid, neodecanoic acid, nervonic acid (cis-15-tetracosenoic acid), nonacosanoic acid, nonadecanoic acid, octacosanoic acid, oleic acid, palmitic acid (n-hexadecanoic acid), palmitoleic acid, pelargonic acid (nonanoic acid), pentadecanoic acid, pentacosanoic acid, petroselenic acid, phytanic acid, stearic acid (n-octadecanoic acid), triacontanoic acid, tricosanoic acid, tridecanoic acid, undecanoic acid, and vaccenic acid. C₁₀₋₂₄ fatty acids are preferred, with C₁₂₋₂₀ fatty acids being most preferred. Preferred fatty acids include lauric acid, myristic acid, palmitic acid, stearic acid, oleic acid, linoleic acid, arachidonic acid, and combinations thereof. Stearic acid, however, is a particularly preferred fatty acid.

Fatty alcohols derive from the fatty acids specified above, i.e., the terminal carboxylic acid group —COOH of the fatty acid is replaced with a CH₂OH group. Examples of suitable fatty alcohols (saturated, unsaturated, and polyunsaturated) include behenyl alcohol, cetyl alcohol, elaidyl alcohol, erucyl alcohol, isostearyl alcohol, lauryl alcohol, myristyl alcohol, oleyl alcohol, palmitoleyl alcohol, petroselinyl alcohol, and stearyl alcohol. Fatty alcohols having a C₁₀₋₂₄ fatty acid are preferred, with fatty alcohols having a C₁₂₋₂₀ fatty acid being most preferred. Preferred fatty alcohols include lauryl alcohol, myristyl alcohol, palmityl alcohol, stearyl alcohol, oleyl alcohol, linoleyl acid, arachidonyl alcohol, cetyl alcohol, and combinations thereof. Stearyl alcohol and cetyl alcohol, however, are particularly preferred fatty alcohols.

Fatty esters have the general structure of R¹—C(O)—O—R², wherein at least one of R¹ and R² contains a C₁₀₋₂₄ carbon chain. Exemplary fatty esters include esters of fatty acids, fatty (long-chain alkyl or alkenyl) esters of monohydric alcohols, diols, and polyols, diols and polyols that are both esterified with a fatty acid and substituted with a polyoxyalkylene, polyoxyalkylene fatty acid esters, polyoxyalkylene fatty ethers, and polyglyceryl fatty acid esters, examples of which are provided below.

Esters of fatty acids are fatty acids whose carboxylic acid groups (—COOH) have been replaced with esters —COOR, where R is a long-chain substituent or a lower alkyl group, for example, cetyl lactate, myristyl lactate, lauryl lactate, isostearyl lactate, and stearyl lactate, ethyl lactate, isopropyl myristate, isopropyl palmitate, ethyl linoleate, and isopropyl linoleate;

Fatty (long-chain alkyl or alkenyl) esters of monohydric alcohols, diols, and polyols are alcohols whose hydroxyl groups (—OH) have been replaced with —O—(CO)-L-CH₃ (“fatty”) groups where L is alkylene or alkenylene containing 1-3 double bonds and from about 6-22 carbon atoms. Examples of monohydric alcohols include fatty alcohols, as discussed above, monohydric sterols such as cholesterol, and lower alcohols (i.e., alcohols containing less than 10 carbon atoms) such as n-propanol, isopropanol, n-butanol, isobutanol, phenol, benzyl alcohol, phenyl ethanol, menthol, 1-dodecanol, and lactic acid. Examples of diols and polyols (generally C₂-C₂₄) include: glycerol; butane diol; alkylene glycols such as propylene glycol, ethylene glycol, diethylene glycol, polyethylene glycol, and polypropylene glycol; monomeric polyols such as trimethylol ethane, trimethylol propane, trimethylol butane, pentaerythritol and dipentaerythritol; sugar alcohols containing 5-12 carbon atoms such as sorbitol or mannitol; and sugars containing 5-12 carbon atoms such as glucose or sucrose. Specific examples of nonionic surfactants within this group are as follows: methyl laurate, ethyl oleate, isopropyl n-decanoate, isopropyl myristate, isopropyl palmitate, sucrose monooleate, cholesterol stearate, octyldodecyl myristate, propylene glycol dilaurate, propylene glycol monooleate, propylene glycol dioctanoate, propylene glycol dicaprylate, propylene glycol dicaprate, glycerol monolaurate, glycerol monooleate, glycerol monostearate; the sorbitan fatty acid esters sorbitan monopalmitate, sorbitan monooleate, sorbitan dioleate, sorbitan trioleate, sorbitan sesquioleate, sorbitan isostearate, sorbitan diisostearate, sorbitan tristearate, and sorbitan monolaurate; and the sucrose fatty acid esters sucrose monooleate, sucrose monostearate, sucrose monolaurate, sucrose distearate, sucrose dipalmitate, and sucrose monopalmitate.

Diols and polyols that are both esterified with a fatty acid and substituted with a polyoxyalkylene, are compounds that include, without limitation, polyoxyethylene and polyoxypropylene glyceryl stearate, laurate, and palmitate, e.g., polyethylene glycol (PEG)-20 glyceryl stearate, polypropylene glycol PPG-10 glyceryl stearate, PEG-15 glyceryl laurate, PEG-20 glyceryl laurate, PEG-30 glyceryl laurate, PEG-40 sorbitol septaoleate, PEG-40 glyceryl laurate; and polyoxyethylene sorbitan fatty acid esters (polysorbates) and polyoxypropylene sorbitan fatty acid esters. Polyoxyethylene sorbitan esters include sorbitan oleates, palmitates, and stearates, e.g., polyoxyethylene sorbitan monolaurate (for example, PEG-20 sorbitan monolaurate, available commercially under the trade name Tween®-20), polyoxyethylene sorbitan monopalmitate (for example, PEG-20 sorbitan monopalmitate, available commercially under the trade name Tween®-40), polyoxyethylene sorbitan monostearate (for example, PEG-20 sorbitan monostearate, available commercially under the trade name Tween®-60), polyoxyethylene sorbitan monooleate (for example, PEG-20 sorbitan monooleate, available commercially under the trade name Tween®-80), polyoxyethylene sorbitan trioleate (e.g., PEG-20 sorbitan trioleate), polyoxyethylene sorbitol septaoleate, and polyoxyethylene sorbitan monooleate. Polyoxypropylene sorbitan fatty acid esters include, by way of example, polyoxypropylene sorbitan monooleate, polyoxypropylene sorbitan monopalmitate, polyoxypropylene sorbitan trioleate, and polyoxypropylene sorbitol septaoleate.

Polyoxyalkylene fatty acid esters are compounds such as polyoxyethylene fatty acid esters and polyoxypropylene fatty acid esters, for example, polyethylene glycol (PEG)-2 stearate, PEG-2 oleate, PEG-4 stearate, PEG-4 laurate, PEG-4-ricinoleate, PEG-4 dioleate, PEG-4 distearate, PEG-4 dilaurate, PEG-6 stearate, PEG-6 dioleate, PEG-6 distearate, PEG-6 dilaurate, PEG-8 stearate, PEG-8 laurate, PEG-8 oleate, PEG-8 dilaurate, PEG-8 dioleate, PEG-8 distearate, PEG-10 stearate, PEG-10 laurate, PEG-10 oleate, PEG-10 dipalmitate, PEG-10 distearate, PEG-12 stearate, PEG-12 oleate, PEG-12 ricinoleate, PEG-12 distearate, PEG-12 dilaurate, PEG-12 dioleate, PEG-20 stearate, PEG-20 oleate, PEG-20 dilaurate, PEG-20 dioleate, PEG-20 distearate, PEG-25 stearate, PEG-30 stearate, PEG-30 cholestanol, PEG-32 oleate, PEG-32 laurate, PEG-32 stearate, PEG-32 dilaurate, PEG-32 dioleate, PEG-32 distearate, PEG-40 laurate, PEG-40 oleate, PEG-40 stearate, PEG-400 dioleate, and PEG-400 distearate;

Polyoxyalkylene fatty ethers are condensation products of alkylene oxides and fatty alcohols, such as diethyleneglycol lauryl ether (PEG-2-L), PEG-24 cholesterol ether, PEG-2 oleyl ether, PEG-3 oleyl ether, PEG-5 oleyl ether, PEG-10 oleyl ether, PEG-20 oleyl ether, PEG-4 lauryl ether, PEG-9 lauryl ether, PEG-23 lauryl ether, PEG-2 cetyl ether, PEG-10 cetyl ether, PEG-20 cetyl ether, PEG-2 stearyl ether, PEG-10 stearyl ether, PEG-20 stearyl ether, and PEG-100 stearyl ether.

Polyglyceryl fatty acid esters include compounds such as polyglyceryl-2 stearate, polyglyceryl-2 oleate, polyglyceryl-2 isostearate, polyglyceryl-3 oleate, polyglyceryl-4 oleate, polyglyceryl-4 stearate, polyglyceryl-6 oleate, polyglyceryl-10 laurate, polyglyceryl-10 oleate, polyglyceryl-10 stearate, polyglyceryl-6 ricinoleate, polyglyceryl-10 linoleate, polyglyceryl-6 pentaoleate, polyglyceryl-3 dioleate, polyglyceryl-3 distearate, polyglyceryl-4 pentaoleate, polyglyceryl-6 dioleate, polyglyceryl-2 dioleate, polyglyceryl-10 trioleate, polyglyceryl-10 pentaoleate, polyglyceryl-10 septaoleate, polyglyceryl-10 tetraoleate, polyglyceryl-10 decaisostearate, and polyglyceryl-10 decaoleate.

Suitable long-chain fatty amines and amides, include dodecyl-N,N-dimethylamino acetate, dodecyl 2-(N,N)-dimethylamino propionate, dimethyl formamide, N,N-diethyl-3-methylbenzamide, dimethyl acetamide, hexamethylene lauramide, higher N,N-dimethylalkylamides (e.g., N,N-dimethylhexanamide, N,N-dimethyloctanamide, N,N-dimethyldecanamide, N,N-dimethyldodecanamide, and N,N-dimethyltetradecanamide), and dimethyl lauramide.

Bile salts are basic addition salts of bile acids, in which the acidic functionality is ionized and associated with a cationic counter-ion, e.g., sodium, potassium, ammonium, or the like. Examples of suitable bile salts include, but are not limited to, sodium cholate, sodium taurocholate, sodium glycocholate, sodium deoxycholate, sodium taurodeoxycholate, sodium glycodeoxycholate, sodium ursodeoxycholate, sodium chenodexoycholate, sodium taurochenodeoxycholate, sodium taurochenodeoxycholate, and sodium N-methyl taurocholate.

Suitable anionic surfactants include, without limitation, long-chain alkyl sulfonates, carboxylates and sulfates, as well as alkyl aryl sulfonates and the like. Preferred anionic surfactants include, by way of example, sodium n-dodecyl sulfate, dialkyl sodium sulfosuccinates (e.g., sodium bis-(2-ethylhexyl)-sulfosuccinate), sodium lauryl sulfate, sodium 7-ethyl-2-methyl-4-dodecyl sulfate, lithium n-dodecyl sulfate, sodium dodecylbenzene sulfonate, sodium oleate, sodium caprate, sodium laurate, sodium lauryl sarcosinate, sodium dioctyl sulfosuccinate, sodium caproate, sodium caprylate, sodium myristate, sodium myristolate, sodium palmitate, sodium palmitoleate, sodium ricinoleate, sodium linoleate, sodium linolenate, sodium stearate, sodium tetradecyl sulfate, sodium lauryl sarcosinate, and sodium dioctyl sulfosuccinate (sodium docusate).

Suitable cationic surfactants are generally long-chain amine salts or quaternary ammonium salts, wherein the quaternary ammonium salts are typically selected from mono C₆₋₁₆, preferably C₆₋₁₀, N-alkyl, or alkenyl ammonium surfactants wherein remaining N positions are substituted by methyl, hydroxyethyl, or hydroxypropyl groups. Examples of cationic surfactants include, without limitation, decyltrimethylammonium bromide, dodecyltrimethylammonium bromide, hexadecyltriammonium bromide, tetradecyltrimethylammonium bromide, tetradecyltrimethylammonium chloride, dodecylammonium chloride, alkyl benzyldimethylammonium chlorides, diisobutyl phenoxyethoxydimethyl benzylammonium salts, and alkylpyridinium salts.

Amphoteric surfactants are generally, although not necessarily, compounds that include a carboxylate or phosphate group as the anion, and a charged amino or quaternary ammonium moiety as the cation. Betaines are amphoteric surfactants that are particularly useful herein, and include cocodimethyl carboxymethyl betaine, cocoamidopropyl betaine, cocobetaine, lauryl amidopropyl betaine, oleyl betaine, lauryl dimethyl carboxymethyl betaine, lauryl dimethyl α-carboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, and the sulfobetaines cocodimethylsulfopropyl betaine, stearyl dimethylsulfopropyl betaine, lauryl dimethylsulfoethyl betaine, and lauryl bis-(2-hydroxyethyl)sulfopropyl betaine. Other amphoteric surfactants include, for example, lauryldimethylamine oxide, n-dodecyl-N,N-dimethylamino glycine, and 3-dodecyl-dimethylammoniopropane-1-sulfonate.

Volatilization of the coating material is carried out according to conventional methods for volatilizing materials. As is known to one of ordinary skill in the art, the process of volatization follows well-established rules of thermodynamics, and takes place by increasing the temperature of and/or by decreasing the pressure surrounding the material to be volatilized. Although simply increasing temperature or decreasing pressure may suffice for volatilizing a given material, a relatively smaller increase in temperature or a relatively smaller decrease in pressure is necessary when both approaches are used for some materials. That is, a correlation exists between temperature and pressure when volatilizing a material, evidenced by the familiar phenomenon of being able to boil water (i.e., to convert liquid water into gaseous water) at a lower temperature at higher elevations where atmospheric pressure is lower.

Although volatilization may take place by increasing the temperature, decreasing the pressure, or both, a preferred method for volatization is by increasing the temperature of the material itself. Consequently, it is useful to know the amount of energy, in the form of heat, required to vaporize (i.e., volatilize) a unit of liquid at its boiling point with no change in temperature. This value, known as the heat of vaporization (ΔHᵥₐₚ), represents the amount of energy required to overcome the intermolecular forces, e.g., van der Waals forces and hydrogen bonding, that maintain the material in a liquid state. The heat of vaporation (ΔHᵥₐₚ) for a given material is provided in the literature or can be calculated using the Clausius-Clapeyron equation (equation I) once the vapor pressure at two different temperatures is established experimentally:

$$ln ( \frac{P}{P°} ) = \frac{Δ {H}_{vap}}{R} ( \frac{1}{T°} - \frac{1}{T} ) \qquad ( I )$$

where P is the vapor pressure of the material at a first known temperature T, P° is a vapor pressure of the material at a second known temperature T°, and R is the ideal gas law constant (8.31434 J mol⁻¹ K⁻¹). As P°, T°, P, T, and R can be determined through routine experimentation, determination of the heat of vaporization for a material is simply a matter of substituting the determined values of P°, T°, P, T, and R into equation (I) and solving for ΔHᵥₐₚ. A relatively high heat of vaporization indicates that relatively more heat is required to convert the material from a liquid to a gas. Conversely, a material with a lower heat of vaporization requires less heat for conversion from a liquid to a gas. Examples of heats of vaporization (ΔHᵥₐₚ) for selected coating materials are provided in Table 1 (CRC Handbook of Chemistry and Physics, 63^{rd} Edition, Boca Raton, Fla.: CRC Press, Inc., 1984).

TABLE 1
Heats of Vaporization for Selected Coating Materials

| Coating Material | Type of Coating Material | ΔHᵥₐₚ (g cal mol⁻¹) |
|---|---|---|
| Stearic acid | Fatty acid | 19,306.6 |
| Oleic acid | Fatty acid | 20,326.7 |
| Palmitic acid | Fatty acid | 17,603.6 |
| Myristic acid | Fatty acid | 18,380.1 |
| Tridecanoic acid | Fatty acid | 19,214.8 |
| Lauric acid | Fatty acid | 16,585.3 |
| Capric acid | Fatty acid | 19,372.6 |
| Cetyl alcohol | Fatty alcohol | 14,483.4 |
| Methyl palmitate | Fatty ester | 17,003.5 |
| Methyl myristate | Fatty ester | 16,051.0 |
| Methyl laurate | Fatty ester | 14,853.5 |

As appreciated by those of ordinary skill in the art, a number of methods are available for heating the coating material for volatilization purposes. Such methods include, for example, using an electrical hot plate, steam, a stove or Bunsen burner, applying microwave or electromagnetic radiation, and so forth. Stearic acid, for example, will melt at about 69-70° C. and slowly volatilizes at a temperature in the range of about 90-100° C. at normal atmospheric pressure.

Alternatively, or in addition to increasing the pressure, the pressure surrounding coating material can be decreased in order to effect volatilization. A number of approaches can be used to decrease the pressure surrounding the coating material. For example, specialized chambers equipped with vacuum pumps can be used to degas the chamber, thereby reducing the pressure surrounding the coating material. In addition, heated gas, typically air, along with the coating material can be placed in a heated chamber, whereupon a reduction in pressure is effected as the air cools. Furthermore, conducting the coating procedure at a higher elevation decreases the pressure surrounding the coating matter. If necessary, both approaches, i.e., reducing pressure and increasing temperature, can be used to ensure volatilization of the material.

Once volatilization takes place, the volatilized coating material is allowed to contact and condense upon the surface of the drug-containing particles. There are a number of approaches for contacting a volatilized material with a drug-containing particle, and the invention is not limited in this regard. For example, the volatilized coating material can be directed, e.g., with the use of a fan, to come into contact with the drug-containing particle. In addition, the volatilized coating material will often come into contact with the drug-containing particle as the gaseous material diffuses through the atmosphere. Optimally, the drug-containing particles are positioned so as to maximize contact with the volatilized coating material.

Upon contact with drug-containing particles, the volatilized coating material condenses onto the particles. Condensation occurs when there is insufficient energy to maintain the volatilized coating material in a gaseous state. Specifically, condensation onto the particles is accomplished by increasing the pressure or decreasing the temperature. In the present context, condensation onto the drug-containing particles can, therefore, take place either by increasing the pressure surrounding the volatilized coating material, or by decreasing the temperature of the volatilized material. Condensation onto the drug-containing particles (as opposed to other surfaces) is effected since the volatilized material is in direct contact with the drug-containing particles. While other surfaces will inevitably be coated as condensation occurs, those surfaces can be cleaned once the coated drug-containing particles have been recovered.

Increasing the pressure surrounding the volatilized coating material can be accomplished using art-known methods. For example, pumps can be used to force air into a closed chamber that contains the volatilized material, thereby increasing the pressure surrounding the volatilized coating material. Other techniques to increase the surrounding pressure can be employed as well.

As the addition of heat is generally used, at least in part, to volatilize the coating material, the reduction heat is a preferred means by which to condense the volatilized coating material. When the temperature of the particle is lower than the temperature of the volatized material, the volatilized material condenses onto the particle. Thus, any approach used to ensure that the temperature of the particle is lower than the temperature of the volatilized material can be used to accomplish condensation.

One technique involves cooling the particles themselves by placing a cooling jacket over the apparatus. As is known by those of ordinary skill in the art, a cooling jacket cools an apparatus, as well as the particles contained therein, by circulating relatively cold fluid or air through the jacket. In this way, the drug-containing particles are maintained at a temperature lower than the volatilized material such that condensation of the volatilized material onto the drug-containing particle occurs. Other methods for maintaining the drug-containing particles at a relatively “cool” temperature relative to the temperature of the volatilized material include freezing or chilling the drug-containing particles.

As its name implies, condensation results in a condensed form of matter, i.e., matter that is in a liquid or solid form. Often, condensation of the volatilized coating material results in a particle coating that is liquid or semi-liquid. If the condensed form of the coating material on the drug-containing particle is in a liquid or semi-liquid form, further cooling of the coated drug-containing particle can be carried out to obtain a solid coating. If the condensed form of the volatilized coating material on the particle is a solid, no further cooling is required.

In addition, while the volatized coating material is still in a tacky state, fine particles of an additional material (e.g., excipients, drug, etc.) can be introduced into the coating chamber to further build up the coating layer. Introduction of such additional materials can be done by adding the materials directly to the coating chamber of the materials can be added to the incoming vaporized stream.

As will be appreciated by one of ordinary skill in the art, the previously described approaches for volatilizing the coating material, thus ensuring contact between the volatilized material and the drug-containing particles as well as condensation of the volatilized material, are merely exemplary in nature. Each approach can be modified, adapted, and/or combined with other methods sufficient to carry out these steps. Modifications to the disclosed approaches, as well as additional approaches not disclosed herein, will be readily apparent to one of ordinary skill in the art and are encompassed within the scope of the invention. Consequently, the disclosed methods do not limit the invention in any way.

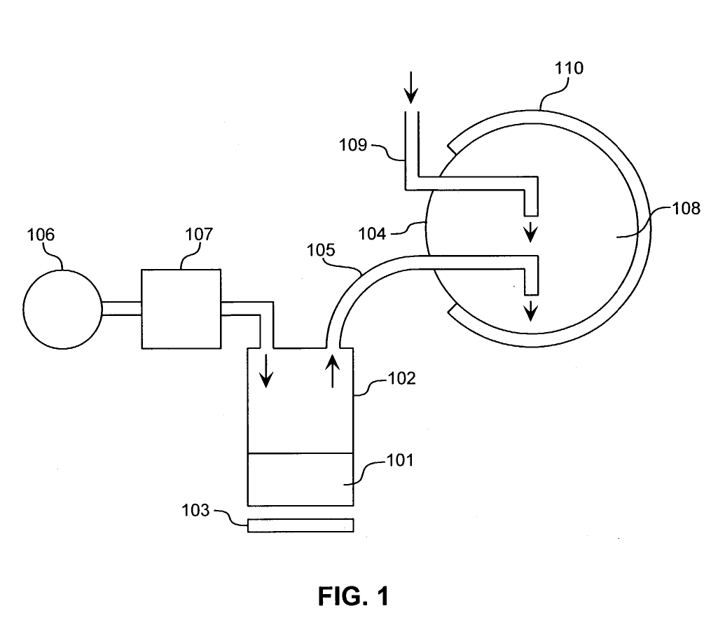

One approach for contacting the volatilized material with the drug-containing particles is to use a coating pan, an example of which is illustrated in FIG. 1. The coating material 101 is placed in a vaporization chamber 102 and heated by heat source 103. The volatized coating material then moves into the coating chamber 104, through a jacketed line 105. A fan 106 and heater 107 provide for continuous flow of warmed gas (e.g., air) air into the vaporization chamber 102 to ensure movement of the volatized coating material. The drug-containing particles 108 are housed in the coating chamber 104, which is agitated or moved continuously in a circular direction during the coating process to ensure that the drug-containing particles are constantly in motion. The continuous flow of the volatized coating material into the coating chamber thus effects contact with the drug-containing particles and cooling air from an external source (not shown) is directed into the coating chamber though an inlet line 109 so as to facilitate condensation of the coating material onto the surface of the drug-containing particles. The coating chamber is also fitted with a cooling jacket 110 to maintain this lower temperature environment.

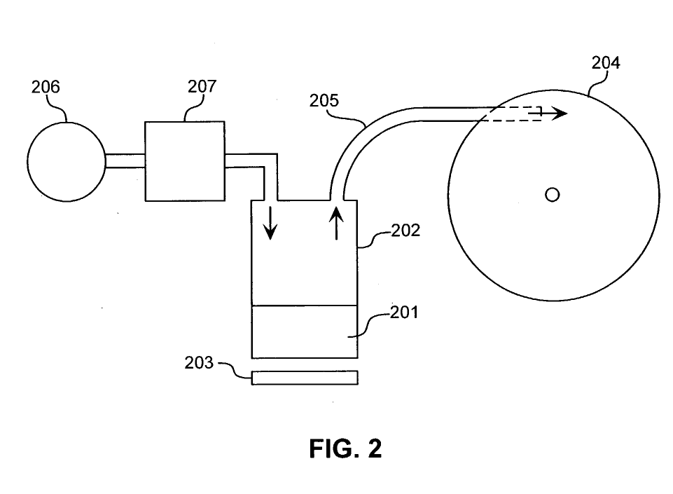

Another for contacting the volatilized material with the drug-containing particles is to use a rotating granulator, an example of which is illustrated in FIG. 2. The coating material 201 is placed in a vaporization chamber 202 and heated by heat source 203. The volatized coating material then moves into the coating chamber 204, through a jacketed line 205. A fan 206 and heater 207 are also provided. The coating chamber 204, which houses the drug-containing particles, is rotated continuously during the coating process to ensure that the drug-containing particles are constantly in motion.

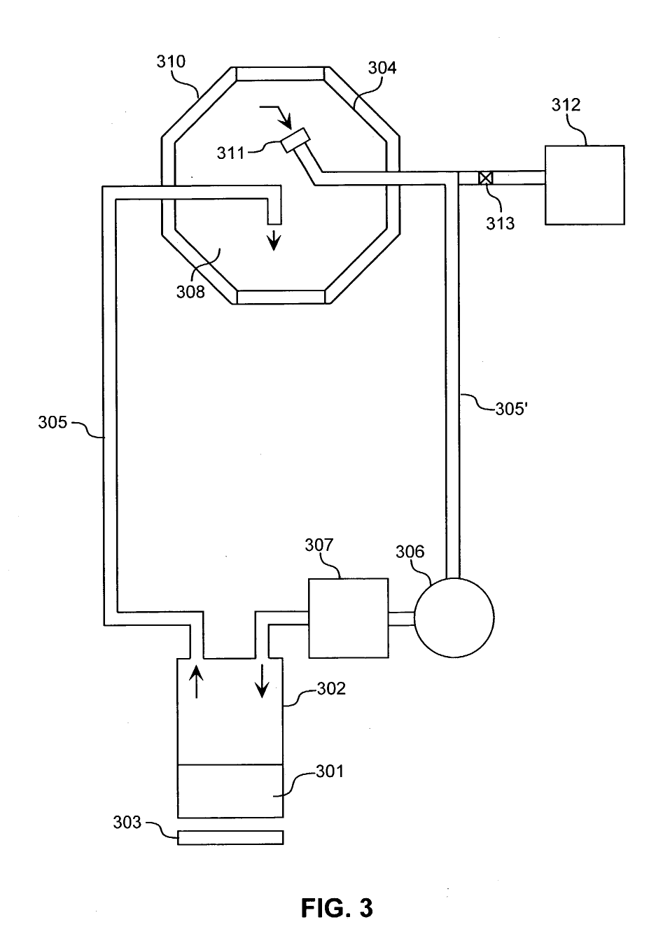

In yet another approach, a tumble-blender (e.g., a V-blender or conical blender) is used, an example of which is illustrated in FIG. 3. The coating material 301 is placed in a vaporization chamber 302 and heated by heat source 303. The volatized coating material then moves into a tumble-blender that has been modified with an inlet for introduction of the volatilized coating material, shown in FIG. 3 as coating chamber 304 and jacketed line 305. A fan 306 and heater 307 provide for continuous flow of warmed air into the vaporization chamber 302 to ensure movement of the volatized coating material. The drug-containing particles 308 are housed in the coating chamber 304, which is rotated continuously during the coating process to keep the drug-containing particles in motion. Contact between the volatilized coating material and the drug-containing particles is effected when the coating material is directed through the inlet line 305. The coating chamber is also fitted with a cooling jacket 310, which typically serves to maintain a temperature below the condensing temperature of the volatized coating material. Volatilization is also facilitated by a decrease in pressure in the coating chamber. This is accomplished by drawing gas from the coating chamber through an outlet jacketed line 305′, which leads into the vaporization chamber. The outlet jacketed line 305′ is fitted with a filter 311 at its opening to prevent removal of any drug-containing particles, a vacuum pump 312 and pressure valve 313.

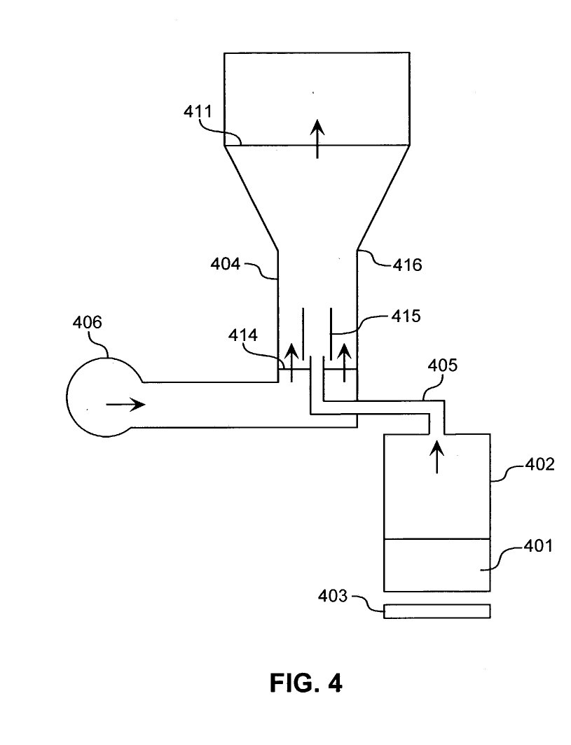

The drug-containing particles can also be contacted by the volatilized material by means of a fluid bed coater, an example of which is illustrated in FIG. 4. This approach involves suspension of the drug-containing particles in a fluidized bed, and then introducing the volatilized coating material through inlets such that the material is allowed to contact the particles. The coating material 401 is placed in a vaporization chamber 402 and heated by heat source 403. The volatized coating material then moves into the coating chamber 404, through a jacketed line 405. A fan 406 provides for continuous flow of cooled gas (e.g., air) into the coating chamber 404 to facilitate condensation of the coating material onto the surface of the drug-containing particles when the particles are in the coating zone 415 and to ensure movement of the drug-containing particles when the particles are in the cooling zone 416. The coating chamber is also provided with a filter 411 and screen 414 to retain the drug-containing particles.

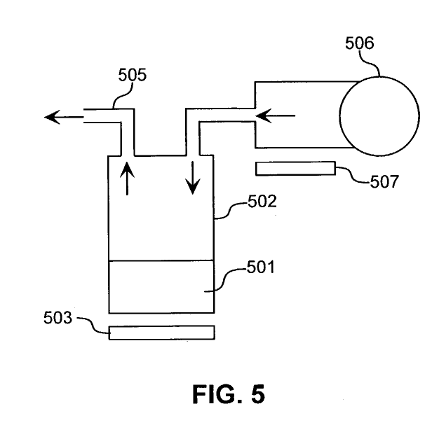

A modification of the fluid bed coater of FIG. 4. is illustrated in FIG. 5. As in the fluid bed coater of FIG. 4, the coating material 501 is placed in a vaporization chamber 502 and heated by heat source 503. The volatized coating material then moves into the coating chamber through a jacketed line 505. However, this embodiment also includes a fan 506 and heater 507 to provide for continuous flow of warmed gas (e.g., air) air into the vaporization chamber 502 to ensure movement of the volatized coating material.

Another approach for maximizing contact includes placing the drug-containing particles on a screen with mesh openings that are smaller than the diameter of the particle. The screen is then positioned above the source providing the volatilized coating material. In this way, the drug-containing particles are allowed to contact the volatilized coating material as it passes through the mesh openings. The screen can be agitated or rotated to allow movement of the drug-containing particles, thereby ensuring that the volatilized coating material contacts all areas on the drug-containing particle.

The invention is not limited with respect to the drug contained in each particle. Each particle can contain only a single drug, or it can contain a combination of two or more drugs. Furthermore, each particle in a pharmaceutical formulation can contain a plurality of drug-containing particles that can be the same, i.e., a formulation comprising particles containing the same drug; or each particle in a pharmaceutical formulation can contain two or more different types of drug-containing particles (for example, a formulation comprising a plurality of particles containing a first drug in combination with a plurality of particles containing a second drug).

The drug in the drug-containing particle can be selected from the following nonlimiting examples:

analgesic agents such as acetominophen, codeine, morphine, diacetylmorphine, hydromorphone, oxymorphone, levorphanol, levallorphan, hydrocodone, oxycodone, nalorphine, butorphanol, nalbuphine, tramadol, meperidine, fentanyl, sufentanil, alfentanil, remifentanil, methadone, levomethadyl, propoxyphene, pentazocine, buprenorphine, meptazinol and dezocine;

antibiotics such as alatrofloxacin, amikacin, ampicillin, amoxicillin, atovaquone, azithromycin, azlocillin, aztreonam, bacampicillin, bacitracin, carbenicillin, cefaclor, cefadroxil, cefamandole, cefazolin, cefepime, cefixime, cefonicid, cefoperazone, ceforanide, cefotaxime, cefotetan, cefoxitin, cefpirome, cefpodoxime, cefprozil, ceftazidime, ceftizoxime, ceftriaxone, cefuroxime, cephalexin, cephalothin, cephradine, chloramphenicol, chlortetracycline, cinoxacin, ciprofloxacin, clarithromycin, clinafloxicin, clindamycin, clofazimine, cloxacillin, colistin, cycloserine, dalfopristin, dapsone, demeclocycline, dicloxacillin, doxycycline, enoxacin, enrofloxacin, erythromycin, ethambutol, ethionamide, fleroxacin, gatifloxacin, gentamicin, grepafloxacin, imipenem, isoniazid, kanamycin, levofloxacin, linezolid, lomefloxacin, loracarbef, mafenide, meropenem, methacycline, methenamine, methicillin, mezlocillin, minocycline, moxifloxacin, nafcillin, nalidixic acid, neomycin, netilmicin, nitrofurantoin, norfloxacin, ofloxacin, oxacillin, oxolinic acid, oxytetracycline, p-aminobenzoic acid, pefloxacin, penicillin, phenazopyridine, piperacillin, polymycin B, pyrazinamide, pyrimethamine, quinupristin, rifabutin, rifampin, rifapentine, sitafloxacin, sparfloxacin, spectinomycin, streptomycin, sulfacetamide, sulfadiazine, sulfadoxine, sulfamethoxazole, sulfanilamide, sulfasalazine, sulfisoxazole, teicoplanin, tetracycline, ticarcillin, tobramycin, trimethoprim, trovafloxacin, and vancomycin;

antifungal agents such as amphotericin B, fluconazole, flucytosine, griseofulvin, itraconazole, ketoconazole, miconazole, nystatin, and terbinafine;

antiviral agents such as acyclovir, adefovir, amantadine, cidofovir, clevudine, docosanol, emtricitabine, beta-L-deoxythymidine, entecavir, famciclovir, fomivirsen, foscarnet, fomivirsen, ganciclovir, idoxuridine, imiquimod, lamivudine, lobucavir, maribavir, oseltamivir, penciclovir, pleconaril, ribavirin, rimantadine, trifluridine, valacyclovir, valganciclovir, vidarabine, and zanamivir;

antiretroviral agents such as abacavir, amprenavir, capravirine, delavirdine, didanosine, efavirenz, emlvirine, indinavir, lopinavir, nelfinavir, nevirapine, ritonavir, saquinavir, stavudine, tenofovir, tipranavir, zalcitabine, and zidovudine;

anesthetic agents such as etomidate, propofol, and ketamine;

antigout agents such as allopurinol, benzbromarone, colchicine, probenecid, and sulfinpyrazone;

anti-Alzheimer's drugs such as choline, donepezil, galantamine, lecithin, physostigmine, rivastigmine, and tacrine;

antiasthma agents such as albuterol, aminophylline, bitolterol, colterol, dobutamine, enprofylline, ephedrine, epinephrine, ethylnorepinephrine, fenoterol, formoterol, isoetharine, isoproterenol, metaproterenol, montelukast, pirbuterol, procaterol, ritodrine, salmeterol, terbutaline, theophylline, zafirlukast, and zileuton;

anticancer agents such as altretamine, aminoglutethimide, L-asparaginase, 5-azacitidine, bleomycin, busulfan, capecitabine, carboplatin, carmustine, chlorambucil, cisplatin, cladribine, carboplatin, cyclophosphamide, cytarabine, dacarbazine, dactinomycin, daunorubicin, docetaxel, doxorubicin, edatrexate, epirubicin, etoposide, floxuridine, fludarabine, 5-fluorouracil, gemcitabine, hydroxyurea, idarubicin, ifosfamide, imatinib, irinotecan, lometrexol, lomustine, mechlorethamine, melphalan, 6-mercaptopurine, methotrexate, mitomycin, mitotane, mitoxantrone, oxaliplatin, paclitaxel, pentostatin, permefrexed, plicamycin, podophyllotoxin, procarbazine, propylthiouracil, raltitrexed, rituximab, semustine, streptozocin, temozolomide, teniposide, thalidomide, thioguanine, thiotepa, topotecan, tositumomab, trastuzumab, trimetrexate, tropotecan, valrubicin, vinblastine, vincristine, vindesine, and vinorelbine;

antiepileptics such as carbamazepine, divalproex, ethosuxamide, ethotoin, fosphenytoin, phenytoin, primidone, gabapentin, lamotrigine, and valproic acid;

antidepressants such as amitriptyline, amoxapine, brofaromine, bupropion, buspirone, citalopram, clomipramine, clorgyline, desipramine, dothiepin, doxepin, duloxetine, fluoxetine, fluvoxamine, gepirone, imipramine, iproniazid, ipsaperone, isocarboxazid, lofepramine, maprotiline, mianserin, milnacipran, mirtazapine, moclobemide, nefazodone, nomifensine, nor₁clomipramine, nor₂clomipramine, norcitalopram, norfluoxetine, norsertraline, nortriptyline, oxaprotiline, pargyline, paroxetine, phenelzine, protriptyline, reboxetine, selegiline, sertraline, tomoxetine, tranylcypromine, trazodone, toloxatone, trimipramine, venlafaxine, viloxazine, and zimelidine;

antidiabetic agents such as acarbose, acetohexamide, chlorpropamide, diazoxide, gliclazide, glimepiride, glipizide, glyburide, metformin, miglitol, nateglinide, pioglitazone, repaglinide, rosiglitazone, tolazamide, tolbutamide and troglitazone;

antidiarrheals such as acetorphan, alosetron, attapulgite, bismuth, difenoxin, diphenoxylate, kaolin, lanreotide, loperamide, octreotide, olycarbophil, psyllium, vapreotide, and zaldaride;

antihelminthics such as albendazole, diethylcarbamazine, ivermectin, mebendazole, metrifonate, niclosamide, oxamniquine, oxantel, piperazine, praziquantel, pyrantel, and thiabendazole;

antihistamines such as acrivastine, astemizole, azatadine, azelastine, brompheniramine, buclizine, burimamide, carbinoxamine, cetirizine, chlorcyclizine, chlorpheniramine, clemastine, clobenpropit, cyclizine, cyproheptadine, dexchlorpheniramine, dimenhydrinate, diphenhydramine, N-ethoxycarbonyl-2-ethoxy-1,2-dihydroquinoline, fexofenadine, hydroxyzine, impromidine, levocabastine, loratidine, meclizine, methdilazine, phenindamine, pheniramine, pyrilamine, promethazine, terfenadine, thioperamide, trimeprazine, tripelennamine, and triprolidine;

antihyperlipidemic agents such as atorvastatin, bezafibrate, cerivastatin, cholestyramine, ciprofibrate, clofibrate, colesevelam, colestipol, ezetimibe, fenofibrate, fluvastatin, gemfibrozil, lovastatin, mevastatin, niacin, pravastatin, and simvastatin;

antihypertensive agents such as acebutolol, amlodipine, atenolol, benazepril, benziodarone, bepridil, betaxolol, bisoprolol, bopindolol, bucindolol, candesartan, captopril, carteolol, carvedilol, celiprolol, clonidine, cromakalim, diazoxide, dilevalol, dipyridamole, doxazosin, enalapril, eprosartan, erythrityl, esmolol, felodipine, flunarizine, fosinopril, gallopamil, guanabenz, guanfacine, guanadrel, hexamethonium, hydralazine, indoramin, irbesartan, isosorbide, isradipine, ketanserin, labetalol, levobunolol, lidoflazine, lisinopril, losartan, mecamylamine, medroxalol, mepindolol, methyldopa, metipranolol, metoprolol, metyrosine, mibefradil, minoxidil, moexipril, nadolol, naphthydrofuryl, nebivolol, nicardipine, nicorandil, nimodipine, nisoldipine, nitrendipine, nitroglycerine, oxprenolol, oxyfedrine, pantaerythritol, penbutolol, perhexeline, perindopril, pinacidil, pindolol, prazosin, propranolol, quinipril, ramipril, ranolazine, reserpine, sodium nitroprusside, sotalol, telmisartan, terazosin, timolol, trandolapril, trapidil, trimetazidine, trimethaphan, urapidil, and valsartan;

antiarrhythmic agents such as adenosine, amiodarone, bretylium, digoxin, digitalis, diltiazem, disopyramide, dofetilide, flecainide, ibutilide, isoproterenol, lidocaine, magnesium, mexiletine, moricizine, nifedipine, phenytoin, procainamide, propafenone, quinidine, tocainide, and verapamil;

antiinflammatory agents such as apazone, aspirin, auranofin, aurothioglucose, celecoxib, choline magnesium trisalicylate, diclofenac, diflunisal, etodolac, fenoprofen, flurbiprofen, gold, ibuprofen, indomethacin, ketoprofen, ketorolac, meclofenamic acid, mefenamic acid, meloxicam, mesalamine, methyl salicylate, nabumetone, naproxen, nimesulide, olsalazine, oxaprozin, piroxicam, rofecoxib, salsalate, sodium salicylate, sulfasalazine, sulindac, and tolmetin;

corticosteroids such as alclometasone, aldosterone, amcinonide, beclomethasone, betamethasone, budesonide, clobetasol, clocortolone, cortisone, dexamethasone, fludrocortisone, flunisolide, fluocinolone, fluocinonide, fluticasone, hydrocortisone, 6-alpha-methylprednisolone, mometasone, prednisone, predinisolone, and triamcinolone;

antimigraine preparations such as ergot alkaloids, ergotamine, methysergide, naratriptan, rizatriptan, sumatriptan. and zolmitriptan;

antinauseants such as dolasetron, domperidone, dronabinol, granisetron, hyoscine, metoclopramide, ondansetron, prochlorperazine, tetrahydrocannabinol, thiethylperazine, trimethobenzamide, and tropisetron;

anti-Parkinsonism drugs such as amantadine, benztropine, bromocriptine, carbidopa, dopamine, entacapone, levodopa, memantadine, pergolide, pramipexole, ropinirole, selegiline, tolcapone, and trihexyphenidyl;

antipsychotics such as amperozide, chlorpromazine, chlorprothixene, cisapride, clopenthixol, clothiapine, clozapine, diethazine, droperidol, epidepride, ethopropazine, eticlopride, flupentixol, fluperlapine, fluphenazine, fluspirilene, haloperidol, loxapine, lithium, melperone, mesoridazine, metiapine, metoclopramide, molindone, nemonapride, olanzapine, oxypertine, penfluridol, perphenazine, piflutixol, pimozide, pipamperone, promazine, quetiapine, raclopride, remoxipride, risperidone, sertindole, sultopride, thioridazine, thiothixene, trifluoperazine, ziprasidone, and zotepine;

antiulcer agents such as Al(OH)₃, CaCO₃, Mg(OH)₂, carbenoxolone, cimetidine, lansoprazole, misoprostol, nazatidine, omeprazole, pantoprazole, pirenzepine, rabeprazole, ranitidine, rebamipide, simethicone, sucralfate, and telenzepine;

appetite suppressants such as amphetamine, diethylpropion, dextramphetamine, fenfluramine, mazindol, methamphetamine, methylphenidate, phenmetrazine, and phenylpropanolamine;

diuretics such as acetazolamide, amiloride, azosemide, bendroflumethiazide, bumetanide, canrenone, chlorothiazide, chlorthalidone, dichlorphenamide, ethacrynic acid, furosemide, hydrochlorothiazide, hydroflumethiazide, indapamide, methazolamide, methyclothiazide, metolazone, muzolimine, piretanide, polythiazide, quinethazone, spironolactone, triamterene, trichlormethiazide, tripamide, and torsemide;

blood clot formation modulators such as abciximab, acenocoumarol, aminocaproic acid, anisindione, antisplasmin, clopidogrel, dicumarol, dipyridamole, eptifibatide, heparin, 4-hydroxycoumarin, indan-1,3-dione, phenprocoumon, phylloquinone, phytonadione, plasminogen, streptokinase, ticlopidine, tirofiban, tissue plasminogen activator, urokinase, and warfarin;

hormone agonists and antagonists such as anastrozole, bicalutamide, bisphenol A, buserelin, cabergoline, carbimazole, cetrorelix, chorionic clomiphene, cyproterone, danazol, desogestrel, gonadotropin, cyproterone, deslorelin, diethylstilbestrol, estradiol, estrone, ethinyl estradiol, estrogen, equilin, exemestane, finasteride, flutamide, fluoxymesterone, follitropin, formestane, ganirelix, genistein, goserelin, gonadotropin-releasing hormone, growth hormone, histrelin, hydroxyprogesterone, letrozole, leuprolide, levothyroxine, liothyronine, liotrix, medroxyprogesterone, megestrol, methyltestosterone, mestranol, methimazole, mifepristone, nafarelin, nilutamide, norethindrone, norgestimate, norgestrel, nortestosterone, octreotide, onapristone, oxandrolone, oxytocin, pegvisomant, progesterone, quinagolide, quinestrol, raloxifene, stanozolol, spironolactone, tamoxifen, testosterone, thyrotropin, thyrotropin-releasing hormone, thyroxine, toremifene, triptorelin, urofollitropin, and vorozole;

immunosuppressive agents such as azathioprine, basilizimab, cyclosporine, daclizumab, infliximab, leflunomide, mycophenolate, sirolimus, and tacrolimus;

muscle relaxants such as atracurium, carisoprodol, curare, decamethonium, doxacurium, mivacurium, pancuronium, pipecuronium, rapacuronium, rocuronium, succinylcholine, tubocurarine, and vecuronium;

narcotic antagonists such as naloxone, nalmefene, and naltrexone;

peptide drugs such as bradykinin, interferon-alpha, interferon-beta, interferon-gamma, interleukin-2, and kallidin;

sedatives and hypnotics such as alprazolam, amobarbital, aprobarbital, barbital, brotizolam, butabarbital, butalbital, chloral hydrate, chlordiazepoxide, clobazam, clomethiazole, clonazepam, clorazepate, demoxepam, diazepam, doxylamine, estazolam, ethchlorvynol, ethinamate, etomidate, flumazenil, flurazepam, glutethimide, halazepam, lorazepam, mephobarbital, meprobamate, methohexital, methyprylon, midazolam, nitrazepam, nordazepam, oxazepam, paraldehyde, pentobarbital, phenobarbital, prazepam, quazepam, secobarbital, sulpiride, temazepam, thiopental, triazolam, zaleplon, zolpicone, and zolpidem;

as well as other drugs such as alendronate, etidronate, metyrapone, aminoglutethimide, trilostane, and mifepristone.

As will be appreciated by one of ordinary skill in the art, any given active agent can be administered for a number of different purposes. Consequently, the categories provided above are merely intended for organizational purposes. In addition, some of the listed active agents are commonly administered in solution. For these active agents, the drug-containing particle can be a liposome or other particle suited for carrying or housing a liquid.

Many active agents have an unpleasant taste. Accordingly, in one preferred embodiment, the coating serves to mask the taste of the drug, thus rendering them more palatable. Exemplary active agents for which taste masking is desirable, include acetaminophen, astemizole, chlorpheniramine, cimetidine, dextromethorphan, erythromycin, famotidine, ibuprofen, loperamide, naproxen, pseudoephedrine, raloxifene, ranitidine, and salts, esters, prodrugs, and combinations thereof, or of any of the foregoing.

The active agent, whether identified above or not, may be administered in the form of a pharmacologically acceptable salt, ester, amide, prodrug, derivative, stereoisomer, or as a combination thereof. Salts, esters, and derivatives of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, acid addition salts are prepared from the free base (e.g., compounds having a neutral —NH₂ or cyclic amine group) using conventional means, involving reaction with a suitable acid. Typically, the base form of an active agent is dissolved in a polar organic solvent such as methanol or ethanol, and the acid is added at a temperature of about 0-100° C., preferably at ambient temperature. The resulting salt either precipitates or may be brought out of solution by addition of a less polar solvent. Suitable acids for preparing the acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like; as well as inorganic acids, e.g., hydrochloric acid, hydrobromic acid, sulfuric acid, nitric acid, phosphoric acid, and the like. An acid addition salt may be reconverted into the free base by treatment with a suitable base. Preparation of basic addition salts of an active agent having an acid moiety (e.g., carboxylic acid group or hydroxyl group) is accomplished in a similar manner using a pharmaceutically acceptable base. Suitable bases include inorganic bases (e.g., sodium hydroxide, potassium hydroxide, ammonium hydroxide, calcium hydroxide, magnesium hydroxide, and the like), as well as organic bases (such as trimethylamine and the like). Preparation of esters involves functionalization of hydroxyl and/or carboxyl groups that may be present within the molecular structure of the drug. The esters are typically acyl-substituted derivatives of free alcohol groups, i.e., moieties that are derived from carboxylic acids of the formula R—COOH, where R is alkyl, preferably lower alkyl, i.e., C₁ to C₆ alkyl. Esters can be reconverted to the free acids, if desired, by using conventional hydrogenolysis or hydrolysis procedures. Preparation of amides and prodrugs can be carried out in an analogous manner. Other derivatives of the active agents may be prepared using standard techniques known to those skilled in the art of synthetic organic chemistry, or may be deduced by reference to the pertinent literature and texts.

Stereoisomers in substantially pure form may also be used. Individual stereoisomers may have unique or beneficial properties that make that individual isomer particularly well suited to be an active agent. Consequently, individual stereoisomers (or mixtures thereof) of the active agents are included. Thus, the active agent may be present in the formulation as a racemate, i.e., in equal amounts of a stereoisomer, a mixture of nonequal amounts of two stereoisomers, or in enantomerically pure form.

The various hydrates of the active agent and other formulation components, if present, may be used. As is known, one or more water molecules may associate with a particular compound based on, for example, the availability of hydrogen bonding. Methods of producing hydrated species are known and include, for example, placing the active agent in a humid environment. In addition, methods of removing one or more water molecules are known and include, by way of example, exposing the active agent to dry heat.

As noted above, the formulation of the present invention may also contain various excipients, provided that such excipients do not have a deleterious effect on the intended patient, or have a deleterious chemical or physical effect on any component in the formulation. Thus, for example, excipients such as preservatives, surface active agents, buffering agents, suspending agents, and the like can be combined with the formulation. The type and amount of any excipient will depend on the type of formulation, the intended route of administration, and other concerns as will be appreciated by one of ordinary skill in the art.

Once coated according to the method described herein, the drug-containing particle can advantageously be used to form a pharmaceutical composition. Generally, a plurality of coated drug-containing particles is used to form the pharmaceutical composition. The composition can be in granulate form, e.g., a granulate used to form compressed tablets. For compositions employed in the formulation of tablets, the drug-containing particles can optionally be combined with one or more of the following excipients: diluents or fillers (e.g., pharmaceutical sugars such as lactose) used to add bulk to the composition; binders (e.g., ethylcellulose, methylcellulose, gelatin, and so forth) used to increase adhesion between formulation components; disintegrants (e.g., sodium alginate) used to promote the breakdown of the tablet; lubricants (e.g., magnesium stearate) used to decrease the adherence of the composition on machine parts; and other excipients such as colors and flavors. The composition can also be used in the filling of capsules. Capsule formulations generally lack binders and disintegrants, but other excipients, e.g., colorants and lubricants, may be included in the formulation. As will be appreciated by one of ordinary skill in the art, the drug-containing particles disclosed herein are appropriate for inclusion in other pharmaceutical formulations that possess excipients suited to that particular type of formulation. Specific examples of other formulations and excipients are well known by those skilled in the art of pharmaceutical formulation.

Also included in the present invention are dosage forms comprising the drug-containing particles described herein. Preferred dosage forms include, without limitation, tablets, capsules, and powders. Preferably the dosage form comprises a formulation as described above. Thus, capsules will house the formulation, and tablets are made by, for example, compressing the formulation using a suitable press apparatus. The dosage form preferably, although not necessarily, will contain a single dose of the active agent or agents.

The invention also includes a method for treating a patient in need of therapy with the active agent, comprising administration of a dosage form that contains a therapeutically effective amount of a formulation as described herein. As indicated above, the dosage form, comprised of the uniquely coated drug-containing particles, can mask the taste of an unpalatable active agent.

The amount of the active agent administered will, of course, be dependent on the particular active agent being used, the patient's age, the patient's weight, and the judgment of the health care practitioner. The amount of the active agent administered in any particular case, however, can be determined by one of ordinary skill in the art based upon routine experimentation and/or reference to the relevant texts and literature.

Typically, the active agent is administered in an amount of from about 1 μg/kg to about 100 mg/kg (amount of drug per kilogram body weight of the patient), more preferably from about 10 μg/kg to about 20 mg/kg. Depending on the patient's response, additional dosages within these ranges can be administered.

It is to be understood that while the invention has been described in conjunction with the preferred specific embodiments thereof, that the foregoing description, as well as the drawings, are intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

What is claimed is:

1. A method for coating drug-containing particles comprising the steps of:
(a) volatilizing a coating material; and
(b) condensing the volatilized coating material onto a plurality of drug-containing particles.

2. The method of claim 1, wherein the volatilizing step is carried out by heating the coating material above its volatilization temperature.

3. The method of claim 1, wherein the condensing step is carried out by maintaining the drug-containing particles at a temperature below the volatilization temperature of the coating material for a time sufficient to effect coating of the drug-containing particles.

4. The method of claim 1, wherein the volatizing step is carried out by decreasing the pressure surrounding the coating material.

5. The method of claim 1, wherein the condensing step is carried out while the plurality of drug-containing particles is agitated.

6. The method of claim 1, wherein the coating material is a lipidic material.

7. The method of claim 6, wherein the lipidic material is selected from the group consisting of long-chain fatty acids, long-chain fatty alcohols, long-chain fatty esters, long-chain fatty amines, and long-chain fatty amides, bile salts, and surfactants.

8. The method of claim 7, wherein the lipidic material is a long-chain fatty acid.

9. The method of claim 8, wherein the long-chain fatty acid is saturated.

10. The method of claim 8, wherein the long-chain fatty acid is unsaturated.

11. The method of claim 8, wherein the long-chain fatty acid is polyunsaturated.

12. The method of claim 8, wherein the long-chain fatty acid contains about 10-24 carbon atoms.

13. The method of claim 12, wherein the long-chain fatty acid contains about 12-20 carbon atoms.

14. The method of claim 13, wherein the long-chain fatty acid is selected from the group consisting of lauric acid, myristic acid, palmitic acid, stearic acid, oleic acid, linoleic acid, arachidonic acid, and combinations thereof.

15. The method of claim 14, wherein the long-chain fatty acid is stearic acid.

16. The method of claim 15, wherein the volatilizing step is carried out by heating the coating material to a temperature within the range of about 90-100° C.

17. The method of claim 15, wherein the volatizing step is carried out by decreasing the pressure surrounding the coating material.

18. The method of claim 15, wherein the volatilizing step is carried out by heating the coating material and decreasing the pressure surrounding the coating material.

19. The method of claim 7, wherein the lipidic material is a long-chain fatty alcohol.

20. The method of claim 19, wherein the long-chain fatty alcohol is saturated.

21. The method of claim 19, wherein the long-chain fatty alcohol is unsaturated.

22. The method of claim 19, wherein the long-chain fatty alcohol is polyunsaturated.

23. The method of claim 19, wherein the long-chain fatty alcohol contains about 10-24 carbon atoms.

24. The method of claim 19, wherein the long-chain fatty alcohol contains about 12-20 carbon atoms.

25. The method of claim 19, wherein the long-chain fatty alcohol is selected from the group consisting of lauryl alcohol, myristyl alcohol, palmityl alcohol, stearyl alcohol, oleyl alcohol, linoleyl alcohol, arachidonyl alcohol, cetyl alcohol, and combinations thereof.

26. The method of claim 25, wherein the long-chain fatty alcohol is cetyl alcohol or stearyl alcohol.

27. The method of claim 5, wherein agitation of the plurality of drug-containing particles is carried out in a blender.

28. The method of claim 27, wherein the maintenance of the temperature of the plurality of drug-containing particles is carried out by controlling the temperature of the blender below the volatilization temperature of the coating material.

29. The method of claim 28, wherein the temperature of the blender is effected by placing a temperature-regulating jacket around the blender.

30. The method of claim 5, wherein agitation of the plurality of drug-containing particles is carried out in a fluidized bed coating apparatus.

31. The method of claim 30, wherein the maintenance of the temperature of the plurality of drug-containing particles is carried out by contacting the drug-containing particles with air at a temperature below the volatilization temperature of the coating material.

32. The method of claim 5, wherein agitation of the plurality of drug-containing particles is carried out in a coating pan.

33. The method of claim 32, wherein the maintenance of the temperature of the plurality of drug-containing particles is carried out by contacting the drug-containing particles with air at a temperature below the volatilization temperature of the coating material.

34. The method of claim 1, wherein the volatilized coating material forms a layer having a thickness of up to 2,000,000 angstroms.

35. The method of claim 34, wherein the volatilized coating material forms a release rate controlling layer.

36. The method of claim 34, wherein the volatilized coating material forms a layer having a thickness of up to 500,000 angstroms.

37. The method of claim 36, wherein the volatilized coating material forms a taste masking layer.

38. The method of claim 34, wherein the volatilized coating material forms a layer having a thickness of up to 10,000 angstroms.

39. The method of claim 38, wherein the volatilized coating material forms a layer that improves flow properties.

40. The method of claim 34, wherein the volatilized coating material forms a layer comprised of a plurality of coating molecules having an average molecular length of no more than 1000 angstroms, and the layer has a thickness of less than about ten times the average molecular length of the coating molecules.

41. The method of claim 40, wherein the coating molecules have an average molecular length of no more than 500 angstroms.

42. The method of claim 40, wherein the layer has a thickness of less than about five times the average molecular length of the coating molecules.

43. The method of claim 1, wherein the active agent is selected from the group consisting of analgesic agents, antibiotics, antifungal agents, antiviral agents, antiretroviral agents, anesthetic agents, antigout agents, anti-Alzheimer's drugs, antiasthma agents, antiepileptics, antidepressants, antidiabetic agents, antidiarrheals, antihelminthics, antihistamines, antihyperlipidemic agents, antihypertensive agents, antiarrhythmic agents, antiinflammatory agents, corticosteroids, antinauseants, anti-Parkinsonism drugs, antipsychotics, antiulcer agents, appetite suppressants, diuretics, blood clot formation modulators, hormone agonists, hormone antagonists, immunosuppressive agents, muscle relaxants, narcotic antagonists, peptide drugs, sedatives, and hypnotics.

44. The method of claim 1, wherein the coating material provides taste masking of the drug.

45. The method of claim 44, wherein the drug in the drug-containing particles is selected from the group consisting of acetaminophen, astemizole, chlorpheniramine, cimetidine, dextromethorphan, erythromycin, famotidine, ibuprofen, loperamide, naproxen, pseudoephedrine, raloxifene, ranitidine, and salts, esters, prodrugs, and combinations thereof, or of any of the foregoing.

46. The method of claim 1, wherein the drug-containing particles are granules, beads, or pellets.

47. The method of claim 1, carried out in the absence of solvents.

48 The method of claim 47, carried out in the absence of methylene chloride.

49. The method of claim 1, carried out at atmospheric pressure at sea level.

50. A composition of matter comprising a drug-containing particle coated with a layer of a coating material comprised of a plurality of coating molecules having an average molecular length of no more than 1000 angstroms, wherein the layer has a thickness of less than about ten times the average molecular length of the coating molecules.

51. The composition of claim 50, wherein the coating material is a lipidic material.

52. The composition of claim 51, wherein the coating material is selected from the group consisting of long-chain fatty acids, long-chain fatty alcohols, long-chain fatty esters, long-chain fatty amines, and long-chain fatty amides, bile salts and surfactants.

53. The composition of claim 52, wherein the lipidic material is a long-chain fatty acid.

54. The composition of claim 53, wherein the long-chain fatty acid is saturated.

55. The composition of claim 53, wherein the long-chain fatty acid is unsaturated.

56. The composition of claim 53, wherein the long-chain fatty acid is polyunsaturated.

57. The composition of claim 53, wherein the long-chain fatty acid contains about 10-24 carbon atoms.

58. The composition of claim 57, wherein the long-chain fatty acid contains about 12-20 carbon atoms.

59. The composition of claim 53, wherein the long-chain fatty acid is selected from the group consisting of lauric acid, myristic acid, palmitic acid, stearic acid, oleic acid, linoleic acid, arachidonic acid, and combinations thereof.

60. The composition of claim 59, wherein the long-chain fatty acid is stearic acid.

61. The composition of claim 52, wherein the lipidic material is a long-chain fatty alcohol.

62. The composition of claim 61, wherein the long-chain fatty alcohol is saturated.

63. The composition of claim 61, wherein the long-chain fatty alcohol is unsaturated.

64. The composition of claim 61, wherein the long-chain fatty alcohol is polyunsaturated.

65. The composition of claim 61, wherein the long-chain fatty alcohol contains about 10-24 carbon atoms.

66. The composition of claim 52, wherein the long-chain fatty alcohol contains about 12-20 carbon atoms.

67. The composition of claim 61, wherein the long-chain fatty alcohol is selected from the group consisting of lauryl alcohol, myristyl alcohol, palmityl alcohol, stearyl alcohol, oleyl alcohol, linoleyl alcohol, arachidonyl alcohol, cetyl alcohol, and combinations thereof.

68. The composition of claim 67, wherein the long-chain fatty alcohol is cetyl alcohol or stearyl alcohol.

69. The composition of claim 50, wherein the active agent is selected from the group consisting of analgesic agents, antibiotics, antifungal agents, antiviral agents, antiretroviral agents, anesthetic agents, antigout agents, anti-Alzheimer's drugs, antiasthma agents, antiepileptics, antidepressants, antidiabetic agents, antidiarrheals, antihelminthics, antihistamines, antihyperlipidemic agents, antihypertensive agents, antiarrhythmic agents, antiinflammatory agents, corticosteroids, antinauseants, anti-Parkinsonism drugs, antipsychotics, antiulcer agents, appetite suppressants, diuretics, blood clot formation modulators, hormone agonists, hormone antagonists, immunosuppressive agents, muscle relaxants, narcotic antagonists, peptide drugs, sedatives, and hypnotics.

70. The composition of claim 50, wherein the coating material provides taste masking of the drug.

71. The composition of claim 70, wherein the drug in the drug-containing particles is selected from the group consisting of acetaminophen, astemizole, chlorpheniramine, cimetidine, dextromethorphan, erythromycin, famotidine, ibuprofen, loperamide, naproxen, pseudoephedrine, raloxifene, ranitidine, and salts, esters, prodrugs, and combinations thereof, or of any of the foregoing.

72. The composition of claim 50, wherein the drug-containing particle are granules, beads, or pellets.

73. A pharmaceutical formulation comprising a plurality of drug-containing particles, each of said drug-containing particles being coated with a layer of a coating material comprised of a plurality of coating molecules having an average molecular length of no more than 1000 angstroms, wherein the layer has a thickness of less than about ten times the average molecular length of the coating molecules.

74. A dosage form comprising a formulation comprised of a plurality of drug-containing particles, each of said drug-containing particles being coated with a layer of a coating material comprised of a plurality of coating molecules having an average molecular length of no more than 1000 angstroms, wherein the layer has a thickness of less than about ten times the average molecular length of the coating molecules.

75. The dosage form of claim 74, in the form of a tablet.

76. The dosage form of claim 74, in the form of a powder.

77. The dosage form of claim 74, wherein the formulation is housed in a capsule.

78. A method of treating a patient comprising the administration of a dosage form containing a therapeutically effective amount of pharmaceutical formulation comprised of a plurality of drug-containing particles, each of said drug-containing particles being coated with a layer of a coating material comprised of a plurality of coating molecules having an average molecular length of no more than 1000 angstroms, wherein the layer has a thickness of less than about ten times the average molecular length of the coating molecules.
